SEQUENCE LISTING (2 sequences)

SEQ ID NO: 1 - protein, 13 aa, Homo sapiens
  MISC_FEATURE (2)..(2): Xaa1 is any amino acid
LXXIXXXXKXXXX

SEQ ID NO: 2 - protein, 15 aa, Homo sapiens
GLNDIFEAQKIEWHE

(19) United States
(12) Patent Application Publication
(10) Pub. No.: US 2009/0088337 A1
(43) Pub. Date: Apr. 2, 2009

(54) RET-Based Analyte Detection

(76) Inventors: Ronald Gill, Denver, CO (US); Miles Brennan, Denver, CO (US); Millard Gambrell Cull, Brighton, CO (US); Anthony West, Denver, CO (US)

Correspondence Address: SWANSON & BRATSCHUN, L.L.C., 8210 SOUTHPARK TERRACE, LITTLETON, CO, 80120, US

(21) Appl. No.: 12/253,743

(22) Filed: Oct. 17, 2008

Related U.S. Application Data
(63) Continuation of application No. PCT/US2007/081865, filed on Oct. 18, 2007.
Application No. PCT/US2007/081865 is a continuation-in-part of application No. 10/888,959, filed on Jul. 9, 2004, now abandoned.
(60) Provisional application No. 60/829,886, filed on Oct. 18, 2006.
(60) Provisional application No. 60/487,018, filed on Jul. 10, 2003.
(60) Provisional application No. 60/509,196, filed on Oct. 6, 2003.

(51) Int. Cl. C40B 30/04; C12Q 1/66; G01N 33/566; C40B 40/10

(52) U.S. Cl. 506/9; 435/8; 435/7.5; 435/7.1; 506/18

(57) ABSTRACT

Compositions and methods for Resonance Energy Transfer (RET) Based Detection of analyte binding are provided.

### REFERENCE TO RELATED APPLICATIONS

This application is a continuation of PCT/US2007/81865, filed Oct. 18, 2007, which application claims the benefit of U.S. Provisional Application Ser. No. 60/829,886, filed Oct. 18, 2006 both of which are entitled “RET Based Antigen Detection,”. This application is also a continuation-in-part of U.S. application Ser. No. 10/888,959, filed Jul. 9, 2004, which application claims the benefit of U.S. Provisional Application Ser. No. 60/487,018 filed Jul. 10, 2003, and also claims the benefit of U.S. Provisional Application Ser. No. 60/509,196, filed Oct. 6, 2003, all of which are entitled “Universal Detection of Binding.” Each of these applications is incorporated herein by reference in their entirety.

### FIELD

This disclosure teaches a Resonance Energy Transfer (RET) Based Detector for analyte binding, and methods of use thereof. The RET Based Detector is a single molecule comprising a Physically Alterable Support, an Energy Donating Reagent and an Energy Accepting Fluorescent Reagent. The Physically Alterable Support comprises a class of antibodies having a modular analyte-binding region, a domain that becomes physically altered upon binding of analyte to the analyte binding site, and, a sequence useful for coupling the binding reagent to a solid support and to the Energy Donating or Energy Accepting Flourescent Reagents. The modular analyte binding region (MABR) is preferably derived from the hypervariable regions of the heavy and light chains of a specific analyte recognizing antibody. The MABR may be introduced into the Physically Alterable Support, as would be understood by one of skill in the art, to create a Detector that may detect a specific analyte of interest. A combination of Energy Donating Reagents and Energy Accepting Fluorescent Reagents are coupled to the Physically Alterable Support. The transfer of resonance energy from the Energy Donating Reagents to the Energy Accepting Fluorescent Reagents is detectably increased upon analyte binding by the MABR of the Physically Alterable Support. This method is useful in any application where detection of analyte binding is desirable, such as diagnostics, research uses and industrial applications.

### BACKGROUND

Immunoassays and assays based on the polymerase chain reaction (PCR) are among the most widely used techniques for detecting analytes. PCR-based techniques generally give very good detection sensitivity and specificity, but are suitable only for applications in which the analyte to be detected is a biological organism (e.g., animal or plant cells, bacteria, viruses and fungal organisms) containing a nucleic acid genome that can be extracted and purified in relatively intact form. PCR-based techniques are not suitable for detecting any of the multitude of analytes that are not nucleic acid-containing organisms (e.g., proteins, polysaccharides, hormones, environmental toxins and pesticides, etc).

Immunoassays are suitable for detecting a much broader range of analytes. They have been adopted in a variety of formats, with enzyme linked immunosorbent assays (ELISAs), lateral-flow immunoassays, and Western-blot assays being the most common. Current ELISA and Western-blot techniques, however, require multiple incubation steps and are prone to operator error. Lateral-flow immunoassays are generally faster but are not as accurate as conventional ELISAs.

Alternative assay formats using some form of resonance energy transfer (RET) to detect specific analytes have been proposed by Tsien, et al. (U.S. Pat. No. 5,998,204), Frommer, et al. (PCT International Application No. 03/025220) and Su, et al. (U.S. Pat. No. 7,247,443).

Tsien, et al. described an analyte detection assay that makes use of a protein having an analyte-binding region that is specific for a particular analyte, and two fluorescent labels. When the analyte-binding region binds the cognate analyte, a conformational change occurs which causes the two fluorescent labels to change position relative to each other. This alters a fluorescence resonance energy interaction between the labels, which is detected in order to determine analyte binding.

Frommer, et al. proposed a similar assay format that makes use of a fusion protein that consists of a bacterial periplasmic binding protein and two fluorescent protein portions. The fusion protein changes conformation upon binding an analyte, changing the relative positions of the two fluorescent protein portions. This alters a fluorescence resonance energy interaction between the labels, which is detected in order to determine analyte binding.

The assay systems disclosed by Tsien, et al., and by Frommer, et al., both require the use of sensor constructs which change conformation upon binding an analyte of interest. Moreover, both detect only small analytes such as simple sugars and amino acids. The practical development of a sensor construct used by either of these methods is dependent upon discovery and adaptation of binding proteins that are naturally occurring in the biological world, and which possess the requisite essential characteristics (i.e., specific binding of the particular analyte of interest and induction of an operationally functional conformational change sufficient to bring the two fluorescent moieties into a suitable juxtaposition to yield an altered RET signal upon binding of the analyte—which is a property of only a relatively small specific subset of binding proteins described in the scientific literature). The potential use of immunoglobulins as flexible, conformationally alterable binding proteins was not envisioned or recognized as being suitable sensors components in either assay system.

The methods described above proposed by Tsein, et al., and by Frommer, et al., each use some form of resonance energy transfer (RET) as an indicator of the presence of analyte. However, each of these methods is severely limited in their applicability by the nature and scope of the analytes that can be detected. Frommer and Tsien require that (a) the analyte be the analyte for a naturally occurring receptor or binding protein, and (b) that the binding protein or receptor undergo a conformational change upon analyte/analyte binding. Not only are the analytes measurable by such methods limited to those for which a naturally occurring binding protein or receptor is known, but in each case the specific conditions for adapting the binding protein or receptor as a RET sensor will be unique.

Su, et al., proposed an alternative analyte detection assay that uses a sensor construct consisting of two fluorescent moieties separated by a rigid, inflexible scaffold. The sensor construct also contains a molecular recognition domain which specifically binds analyte. In contrast to the RET methods discussed above, the sensor described by Su et al. binds analyte without causing a conformational change in the scaffold or positioning of the fluorescent moieties. The two fluorescent moieties possess the requisite fluorescent properties, and are held by the scaffold at an appropriate distance and relative three dimensional positioning, to allow a RET interaction to occur. Binding of the analyte to the molecular recognition domain juxtapositions the bound analyte between the rigidly held donor and acceptor molecules, thereby altering their resonance energy interactions. The observed changes in RET are detected as an indicator of analyte binding.

Su, et al, describe the rigidity of the molecular scaffold component of their sensor used in their method as an absolutely essential property that is necessary for the proper functioning of the sensor. Additionally the rigidity of the scaffold is the critical feature which Su et al., use to distinguish their method from other RET-based methods, such as those described by Tsein and Frommer, in which analyte binding-induced conformational alterations are an essential feature.

The method of Su overcomes the requirement of the previous RET methods to identify a naturally occurring receptor or binding protein that is specific for each analyte to be detected, and which undergoes a suitable conformational change upon binding the analyte. Nevertheless, there are major limitations to the Su method. First, it is likely that molecular size of the analyte will be critically important for proper function. An analyte which is too small may not have sufficient bulk to disrupt the resonance energy interactions between the donor and acceptor moieties. Conversely, analytes that have a very large molecular size, (e.g., bacterial or viral particles and perhaps even large globular proteins) may be too large to gain access to the relatively small distance between the donor and acceptor moieties. In either case the analyte may be inefficiently detected. A second limitation to the method of Su et al., is a common shortcoming of assays that use diminution or disruption of a signal (in this case RET) as a measure of a positive binding event. Such assays are inherently susceptible to false-positive results since many conditions or interfering substances may be present in the sample to be tested which non-specifically interfere with the generation of the RET interaction. Such interference would result in a sensor output that may be indistinguishable from that of bound analyte. These limitations and those of the earlier RET-based assays have been overcome by the innovations incorporated into the methods described herein.

This disclosure provides a detector that exploits the extraordinary properties of the basic structure of IgM conformational change upon binding to analyte such that the Energy Donating Reagent and the Energy Accepting Fluorescent Reagent are brought into juxtaposition generating a resonance energy interaction that can be optically detected. The detection is not limited by the size of the analyte. The analyte binding domain yields virtually unlimited specificities that can be derived in vivo or in vitro. Additionally, there is the flexibility of having a universal platform upon which modular analyte binding regions may be spliced. Modular design of naturally occurring IgM is such that it allows a simple exchange by recombinant DNA methodologies of one analyte binding domain with another of a different specificity.

Antibody molecules are frequently used to detect and quantitate levels of antigen. The binding properties of the antibody reagent in these assays typically impart a high degree of specificity and sensitivity. The challenge of these assays is to detect the binding event. There are several solutions in common practice. The most common method is the use of a “sandwich” assay. In this configuration, the capture antibody is immobilized on a surface and reacted with the unknown sample containing the antigen of interest. Following appropriate wash steps to remove unbound molecules, the bound antigen is detected using a second, antigen-specific antibody. Strategies to visualize the bound second antibody include using a second antibody that has been chemically coupled to a detector, or using a third detection antibody (also chemically coupled to a detector) that specifically recognizes and binds to the secondary antibody of the “sandwich” (e.g., using a mouse capture antibody, a rabbit secondary antibody, and a goat anti-rabbit Ig detection antibody). In the various iterations of this basic assay, the detectors for visualization of the bound antibody include for example, enzymes that react with substrates to give distinctive (e.g., colored) products, fluorescent dyes, or gold particles. The detection may also be by surface plasmon resonance (SPR), in which the increased mass of the bound second antibody is directly measured on a surface by a change in manner in which it reacts with incident reflected light.

For detection of any particular antigen, this basic “sandwich” assay requires 1) two antigen specific antibodies (or proteins showing high, specific binding to antigens) that bind to a single antigen molecule in a non-interfering manner, and 2) and means of detecting the bound secondary antibody/binding protein. The requirement for two antibody proteins that can simultaneously bind a single antigen molecule has limits or complicates the use of this assay format in certain circumstances. It limits the use of this format for detection of antigens, including small monovalent haptens and small peptides (e.g., small peptide hormones). It also complicates the use of this general format for use in proteomics antibody-multiarrays in which the presence of large numbers of antigens is being simultaneously detected. In this case, the need for an antigen-specific secondary antibody doubles the number of antibody reagents that must be developed and significantly compounds problems that arise from the each secondary antibody's particular binding specificities, wash requirements etc.

Presently, the identification of antigen:antibody complexes has taken a number of different forms. As non-limiting examples, 1) an antibody is immobilized and allowed to react with a sample. Bound antigen is then detected by binding of a second, labeled, molecule such as an antigen for the antigen or another antibody directed against another epitope of the antigen; 2) An antibody is immobilized and then allowed to react with a sample. The occupancy of the antigen binding sites by antigen from the sample is determined by a subsequent or concurrent reaction with labeled antigen; 3) an antibody is immobilized on a substrate such as a slide and then allowed to react with a sample. The antigen:antibody complex is detected by a method such as surface plasmon resonance; 4) an antibody in solution is reacted with a sample and with a labeled antigen. The amount of antigen displaces labeled antigen, and the amount of antigen in the sample is reflected in the decreased polarization; and 5) all components of a sample are chemically labeled (e.g., with a fluorescent dye such as Cy3 or Cy5), and then allowed to react with the immobilized antibody. Antigen binding to specific antibody spots is assessed by fluorescence. These techniques all exploit either an antigen specific reagent and/or the molecular weight of the antigen:antibody complex relative to antigen or antibody alone.

For these applications in particular, and for general use with solid phase immunoassays, it would be a significant benefit to utilize a single “universal” assay that detects antigen-antibody binding and that does so in a manner that is not specific for the particular antigen involved.

Most vertebrates produce several isotypes of immunoglobulin (e.g., IgM, IgG, IgA, IgD, IgE) that differ by their heavy chain constant region and have specialized biological properties. The basic immunoglobulin structural unit is composed of four peptide chains, two identical heavy chains and two identical light chains, forming a Y-shaped molecule. Each unit contains two antigen-combining sites (one at each tip of the “Y”). Additional domains on the stem of the “Y” mediate various effector functions such as F_{c}-receptor binding and complement component C1q binding that leads to complement activation via the classical pathway.

IgM is found typically as monomeric, pentameric or hexameric molecules. The pentameric and hexameric forms are respectively composed of five and six of the IgG-like subunits described above. The pentameric form contains an additional single peptide, the J-chain that is lacking in the hexamer. The IgG-like subunits and J-chain (in the case of the pentamer) are held together in the multivalent molecules by inter-chain disulfide bonds. In the absence of bound antigen both the pentamer and hexamer form a flat planar molecule in solution. Upon binding specific antigen, there is a well documented and dramatic conformational change in the molecule that now assumes a “staple” configuration, with nearly a 90-degree angle formed between the F_{ab} and F_{c} portions of the monomer subunits. This conformational change has important biological consequences that are exploited in this disclosure. In particular, the spatial relationship between the variable and constant regions of the antigen bound form of the IgM has been significantly altered. The planar molecule is about 360 Å across and about 40 Å thick. When the IgM is a planar molecule in the non-antigen bound state, its variable regions are about 120 Å from the molecule's center. In the “staple” antigen bound state the IgM variable regions are about 35 Å closer to the molecule's center. A change of this magnitude is amenable to monitoring by Resonance Energy Transfer.

The principle of energy transfer between two molecules can be exploited as a means to provide information about relative changes in their proximity and orientation. Resonance Energy Transfer (RET) is the transfer of excited state energy from a Energy Donating Reagent to an Energy Accepting Fluorescent Reagent. Förster resonance energy transfer (FRET) is a distance-dependent interaction between the electronic excited states of two dye molecules in which excitation is transferred from a Energy Donating Reagent to an Energy Accepting Fluorescent Reagent without emission of a photon. This can only occur if the absorption spectrum of Energy Accepting Fluorescent Reagent overlaps with the fluorescence emission spectrum of the Energy Donating Reagent. Förster determined that the degree of resonance energy transfer between the energy Energy Donating Reagent and Energy Accepting Fluorescent Reagent is inversely proportional to the distance between the two molecules to the sixth power. In the case of FRET, an external light source of specific wavelength is used to excite the donor molecule.

Forster resonance energy transfer is the physical principal utilized in the present methods to detect analytes. FRET involves the transfer of energy from a Energy Donating Reagent, normally a fluorescent or luminescent moiety in its excited state, to another excitable moiety, a Energy Accepting Fluorescent Reagent, by a nonradiative dipole-dipole interaction. Energy Donating Reagent and Energy Accepting Fluorescent Reagent pairs are chosen for the present sensor constructs so as to ensure that a FRET interaction is possible between the donor and Energy Accepting Fluorescent Reagent. The donor's emission spectrum should generally have a shorter wavelength than the Energy Accepting Fluorescent Reagent's emission spectrum (i.e. the donor's optimal emission wavelength maximum, λₘₐₓ, should be shorter than the Energy Accepting Fluorescent Reagent's). In classical FRET, the emission spectrum of the Energy Donating Reagent and the absorption spectrum of the Energy Accepting Fluorescent Reagent must overlap to some extent. There are, however, exceptions to the rule as in the case of anti-Stokes' shift FRET based on lanthanide chelate donors and nonoverlapping Energy Accepting Fluorescent Reagents (see, e.g., Anal. Chem. 2005 Mar. 1; 77(5):1483-7).

In addition, the Energy Donating Reagent and Energy Accepting Fluorescent Reagent must be positioned in close proximity on the sensor construct in order for a FRET interaction to take place. Generally, FRET is seen to occur when the donor and Energy Accepting Fluorescent Reagent are separated by less than about 10 nanometers, but FRET has also been detected at a molecular distance as high as about 25 nanometers between the donor and Energy Accepting Fluorescent Reagent (see, e.g., J. Am. Chem. Soc. 2005 Mar. 9; 127(9):3115-3119). According to Forster's theory, the energy transfer efficiency also depends on the refractive index of the media between the donor and Energy Accepting Fluorescent Reagent, the rate constant for fluorescence/luminescence emission by the energy donor, and the quantum yield of the donor in the absence of the Energy Accepting Fluorescent Reagent. The intensity of an optical signal generated by a FRET interaction between a Energy Donating Reagent and Energy Accepting Fluorescent Reagent of the present sensor construct depends on the identity of the Energy Donating Reagent and Energy Accepting Fluorescent Reagent, as different donor and Energy Accepting Fluorescent Reagent pairs have different Forster radii.

When a Energy Donating Reagent is in the excited state and a FRET interaction takes place between the Energy Donating Reagent and an Energy Accepting Fluorescent Reagent, the fluorescence or luminescence of the donor is quenched (i.e., reduced in total light output). For some Energy Accepting Fluorescent Reagents, light emission is triggered by energy absorbance or transfer from an Energy Donating Reagent, resulting in a light emission by the Energy Accepting Fluorescent Reagent. Other Energy Accepting Fluorescent Reagents, known as quenchers, dissipate the energy absorbed from a Energy Donating Reagent and do not emit light (or do so weakly).

A FRET interaction in the present methods can be detected by measuring the steady-state or time-resolved fluorescence or luminescence of the sensor construct using standard techniques known in the art (see, e.g., Curr. Opin. Chem. Biol. 2003 October; 7 (5):635-40). For example, the fluorescence of the Energy Accepting Fluorescent Reagent or the quenching of Energy Donating Reagent fluorescence of luminescence, or both, can be measured. When a quencher is used as the Energy Accepting Fluorescent Reagent, the quenching of the Energy Donating Reagent's emission is generally measured. In the present methods, the interference with an expected FRET interaction between a Energy Donating Reagent and Energy Accepting Fluorescent Reagent indicates the presence of an analyte in a sample.

Bioluminescent Resonance Energy Transfer (BRET) uses biological molecules such as a luciferase as the Energy Donating Reagent. Luciferases that use coelenterazine as a substrate generate blue light in the range of about 450 to 500 nm depending on the species of origin. When a suitable Energy Accepting Fluorescent Reagent is in close proximity, the blue light energy is captured by RET. The Energy Accepting Fluorescent Reagents are generally a class of proteins that have evolved the ability to be excited by blue light and then fluoresce in longer wavelengths typically with maximal spectral emissions above 500 nm. In both FRET and BRET the Energy Donating Reagent and Energy Accepting Fluorescent Reagents of interest may be either covalently or non-covalently linked or brought in to proximity by conformational change or by spatial migration. For instance Energy Donating Reagents and Energy Accepting Fluorescent Reagents may each be conjugated to two separate proteins of interest. They may then be brought into proximity by their affinity for one another or their affinity for a third molecule. The Energy Donating Reagents and Energy Accepting Fluorescent Reagents may also be attached to a single protein of interest and then brought closer due to a conformational change with in the protein of interest. Generally the Energy Donating Reagents and Energy Accepting Fluorescent Reagents must be within about 100 Å of one another for resonance energy transfer to occur and distance changes as little as about 1-2 Å may be detected. Luciferases that have been used in BRET include those from the firefly, Renilla reniformis and Gaussia princeps. A commonly used fluorescent protein is the green fluorescent protein (GFP) from Aequorea Victoria. The molecular weights of the Gaussia luciferase and GFP are 19.9 kDa and 27 kDa respectively. These molecules are usually sufficiently small enough for to be attached to other proteins of interest without interfering with either their own functions or that of the protein of interest.

All references cited herein are incorporated by reference in their entirety.

### SUMMARY OF THE INVENTION

The present disclosure teaches a general RET Based Analyte Detection system, and methods of use thereof. The system comprises: 1) a Physically Alterable Support, and 2) an Energy Donating Reagent and 3) an Energy Accepting Fluorescent Reagent. The Physically Alterable Support comprises a class of antibodies having an analyte-binding site, a separate domain that becomes physically altered upon binding of analyte to the analyte binding site, and, optionally, a sequence useful for coupling the binding reagent to a solid support. In addition, other sequences may be incorporated into the Physically Alterable Support to facilitate its attachment to the Energy Donating Reagents and the Energy Accepting Fluorescent Reagents.

The Energy Donating Reagent includes molecules that either intrinsically emit light through a chemiluminescent reaction or by reemitting light absorbed from an extrinsic source. These include but are not limited to dyes and proteins. The Energy Accepting Fluorescent Reagent is any molecule that will accept resonant energy emitted by the Energy Donating Reagent and reemit that energy as light. These include but are not limited to dyes and proteins. Upon activation of the Energy Donating Reagent, it and the Energy Accepting Fluorescent Reagent exist in a resonance energy state that is governed by: 1) the degree of overlap between the spectrum of wavelengths that Energy Donating Reagent emits energy at and the absorption spectrum of the Energy Accepting Fluorescent Reagent 2) the distance between the two molecules and 3) the relative dipole-dipole orientation between the two. The Energy Donating Reagent and the Energy Accepting Fluorescent Reagent are contacted to the Physically Alterable Support. Upon analyte binding the resonance energy state between the Energy Donating Reagent and the Energy Accepting Fluorescent Reagent is altered. This change is manifested as an increase in the intensity of light emitted in the spectrum of wavelengths of the Energy Accepting Fluorescent Reagent.

In a preferred embodiment the Physically Alterable Support is an IgM hexamer or pentamer antibody and the analyte is its cognate analyte, in which case the embodiment is a general system for detecting analyte-antibody binding events. The system is based on detection of conformational changes that normally occur when certain antibody molecules bind to their cognate specific analyte. This response is particularly dramatic for IgM, but may also occur to a significant extent with other isotypes. Changes in antibody conformation upon analyte binding are detected by an alteration in the BRET due a change in the proximity and orientation between a bioluminescent protein and a fluorescent protein that are covalently or non-covalently arrayed on the IgM.

This embodiment is useful in any application where detection of analyte binding is desirable, such as diagnostics, research uses and industrial applications. This method is particularly well suited to detecting analyte-antibody complexes on either protein or antibody microarrays, although fluid phase applications using soluble components are also disclosed.

A novel aspect of this embodiment is the development of a single reagent that will detect analyte-antibody binding for multiple antibody species (preferably for antibodies of the IgM isotype) irrespective of their individual analyte specificity. Furthermore the magnitude of the detection signal is expected to be uniform for all antibody-analyte pairs.

The Detector is unique in that it combines the following features in a single molecular platform:
1) An intrinsic Energy Donating Reagent
2) An Energy Accepting Fluorescent Reagent
3) A domain that becomes significantly physically altered upon binding of analyte to the analyte binding site.
4) A modular analyte binding site the amino acid sequence of which may be altered as needed or desired to specifically accommodate a wide spectrum of analytes.

Advantages of this method include:
1) It is a positive assay in that it detects an increase in detectable light.
2) It does not require a wash step after application of the Analyte.
3) It can function as a Detecting element either bound to a surface or floating in solution.
4) It can be modified to accommodate the detection of a wide variety of Analytes by altering the MABR while still retaining the same detection Platform. This makes the assay scalable in that it may be used to simultaneously detect a wide variety of Analytes on a multi-array.
5) It can provide a quantitative signal where the amount of long wavelength light is proportional to the amount of analyte in the sample.

### BRIEF DESCRIPTION OF THE FIGURES

FIG. 1 is a schematic representation of a RET Based Analyte Detector. In this embodiment the Detector is a hexameric form of the IgM that has been modified to have the GFP fused to the carboxyl end of the variable region of light chain and the Avitag™ fused to a linker fused to the carboxyl end of the constant region of the heavy chain. In this planar (non-analyte bound) form of the molecule a single tetramer of streptavidin is bound to one of the 12 biotinylated Avitags™ and resides in the center of the hexamer. A single luciferase with a carboxyl terminal fusion of biotinylated Avitag™ is bound to the streptavidin. The resulting platform or scaffold has 12 GFPs fused to the 12 Light Chain carboxy termini with one streptavidin molecule sitting in the center of the hexamer.

FIG. 2 is the same molecule as in FIG. 1 viewed from the side and with only two arms of the hexamer presented. In this non-analyte bound form the Detector emits blue light from the luciferase in the presence of the Chemiluminescent Substrate and very little or no green light. In this configuration the GFPs are approximately 120\acute{Å} from the center of the IgM.

FIG. 3 represents the analyte bound form of the Detector. Here the Detector undergoes a significant conformational change that brings the GFP approximately 35 \acute{Å} closer to the center of the IgM. This brings the GFPs significantly closer to the luciferase at the center of the IgM. Resonant energy is transferred from the luciferase to the GFP. The Detector thus emits a significantly greater amount of green light in the presence of the Chemiluminescent Substrate due to the transfer of resonance energy from the luciferase to the GFPs.

FIG. 4 is a photograph of a western blot examination of protein expressed by modified IgM transduced cells. Cell supernatants were fractionated on a denaturing 4-12% non-reducing acrylamide gel, transferred to nitrocellulose and probed with goat anti-mouse IgM (μ chain) conjugated to Horse Radish Peroxidase (HRP). The presence of antibody bound protein was visualized with HRP chemiluminescent substrate. Arrow indicates high molecular weight species that is identified by the anti-IgM antibody probe.

FIG. 5 is data from an ELISA antigen capture assay of modified IgM by TNP antigen. Increasing volumes of tissue culture supernatants of wild type IgM Sp6/HL (♦), modified IgM (▪), and negative control sera QB1.293AH (▴) were added to wells of 96 well plate coated with TNP-ovalbumin (antigen). Following blocking of the wells with 3% BSA, all unbound material was washed away and the presence of IgM was detected with goat anti-mouse IgMμ-HRP conjugate. Excess enzyme conjugate was washed off and HRP colorimetric substrate added to well. The plate was incubated for 15 minutes and 2M H₂SO₄ is added and the absorbance at 450 nm was determined.

FIG. 6 represents blotting experiments on the modified expressed IgM to determine if it 1) possesses the L Chain fused to EGFP and 2) is biotinylated as would be expected if the Avitag is fused to the H Chain. Protein from tissue culture supernatants was from pooled and IgM was purified using HiTrap™ IgM Purification HP Column (GE Healthcare). Biotin ligase, biotin, ATP and MgCl₂ were reacted with purified IgM to biotinylate the Avitag™ peptide fused to the heavy chain.

Biotinylated, purified, modified IgM/EGFP/Avitag™ (Lanes B, C, D) and unmodified wild type IgM (Lane A) were fractionated on 4-20% SDS acylamide gels under denaturing and reducing conditions and transferred to nitrocellulose using standard protocols. The nitrocellulose blot (after blocking with PBS and 2% dry milk) was cut into individual strips and probes with indicated probes.

Blots were probed with either rabbit anti-EGFP then goat anti rabbit-HRP, anti-IgM (μ chain) conjugated to HRP, or streptavidin conjugated to alkaline phosphatase (AP) as follows: Lane A) Rabbit anti-EGFP then goat anti-rabbit-HRP and anti-IgM (μ chain) conjugated to HRP; Lane B) Same as A; Lane C) rabbit anti-EGFP then goat anti-rabbit-HRP; Lane D) Streptavidin conjugated to AP. If probe was HRP conjugated, Enhanced Chemiluminescnce (ECL) was used to detect probe. Streptavidin conjugated AP was detected with NBT/BCIP reagent.

FIG. 7 is graph of data demonstrating an increase in longer wavelength light that is directly proportional to the amount of IgM/EGFP in a luciferase catalyzed chemiluminescent reaction. Increasing amounts of either IgM/EGFP (▪) or IgM () were preincubated (5 min) with a fixed amount of Gaussia princeps luciferase (5 ng) and DABCYL-PLUS (Ananspec) dye (375 μg/ml) in 50 μl reaction buffer [50 mM Tris-HCl, (pH 7.8), 600 mM NaCl, 1 mM EDTA, 20% BPER II (Pierce)]. The chemiluminescent reaction was initiated by the addition of 100 μl of 60 μM coelenterazine in PBS. The light output was detected with a Berthold Microplate Luminometer LB964 while integrating over 10 seconds. The units detected are indicated in relative light units.

### DETAILED DESCRIPTION OF THE INVENTION

Unless otherwise defined, all technical and scientific terms used herein have the same meaning as commonly understood by one of ordinary skill in the art. Although methods and materials similar or equivalent to those described herein can be used in the practice or testing of the present invention, suitable methods and materials are described herein. In case of conflict, the present specification, including definitions, will control. In addition, the materials, methods, and examples are illustrative only and are not intended to be limiting.

The disclosure teaches a general method for detecting analyte binding, particularly analyte:antibody complexes. A preferred embodiment comprises three reagents: 1) a Physically Alterable Support with a Modular Analyte Binding Region, and 2) an Energy Donating Reagent and, 3) an Energy Accepting Fluorescent Reagent.

The following terms are intended to have the following general meanings as they are used herein.

### DEFINITIONS

“Analyte” refers to a molecule, compound, or other component in a sample. Analytes include, but are not limited to peptides, proteins, polynucleotides, organic molecules, sugars and other carbohydrates or carbohydrate polymers, lipids, and other types of molecules, including vitamins, hormones, and disease markers. Analytes also include, but are not limited to, large complexes of molecules such as viruses, bacteria or yeast. Analytes also include but are not limited to, chemicals, pesticides and environmental pollutants. Analytes can occur in combination with and/or as portions of other molecules. Analytes also include, but are not limited to, any compound that is able elicit an antigenic immune response from animals or compounds to which a peptide base recognition sequence can be engineered by those knowledgeable in the art. Analyte” is any molecule that is capable of being captured in an antibody-binding site. Analytes preferably do not include RET Energy donors or RET Energy Energy Accepting Fluorescent Reagents.

“Physically Alterable Support” includes a class of antibody constant regions that may be attached to a Modular Analyte Binding Region, a portion that becomes physically altered upon binding of analyte to the Modular Analyte Binding Region, and, optionally, a sequence useful for coupling the binding reagent to a solid support, also known as a tag. The Physically Alterable Support is any monomer or oligomer of immunoglobulin constant regions that, when attached to the Modular Analyte Binding Region (MABR) becomes physically altered when the MABR binds specifically to its cognate analyte. The physical alteration is manifested as a bend in the IgG like portion of the antibody anywhere between the F_{ab} and F_{c} regions. The Physically Alterable Support is typically a modular molecule, including but not limited to fusion (“chimeric”) molecules, that can be attached to a MABR and a portion that becomes physically altered upon binding of analyte to the MABR. In general, this molecule may be naturally occurring or may be made through genetic engineering, but other methods that accomplish the same goals are contemplated. The portion that undergoes physical alteration upon analyte binding can include a wide variety of those currently known in the art, including but not limited to that derived from the Immunoglobulin M (IgM). A preferred portion that undergoes physical alteration upon analyte binding is the constant region of the IgM molecule. Upon analyte binding, the IgM molecule undergoes a physical change that alters the position of the F_{ab} sequences relative to the F_{c} sequences. For the purposes of this invention, IgM can be monomeric, pentameric or hexameric.

“Modular Analyte Binding Region (MABR)” is any specific analyte binding moiety that is attached to the arms of the Physically Alterable Support and that, upon binding either covalently or non-covalently, its cognate analyte, induces a conformational change in the Physically Alterable Support. A non-limiting embodiment for a MABR would be an amino acid based structure that non-covalently binds the analyte. A preferred embodiment of the MABR includes the hypevariable regions of the light and heavy chains of an antibody that when assembled possess the tertiary structure that confers noncovalent binding that is specific to the analyte. In this case the physically alterable region of the RET Based Analyte Detector that undergoes a conformational change resides between the MABR and the constant regions of the antibody.

A preferred MABR is the variable region of an immunoglobulin molecule or F_{ab} sequences of immunoglobulin molecules. The attachment of the MABR to the Physically Alterable Support may by performed by those knowledgeable in the art through the induction of an antigenic response in an animal immunized with the anlayte. In this embodiment, specificity to the analyte results from selection of antibodies that are encoded by sequences that have been hypermutated by rearrangements in their heavy and light chain hypervariable regions. The sequence specificity of the MABR is introduced in to the Physically Alterable Support, in the IgM producing cell through in vivo recombination of the heavy and light chain hypervariable regions of the analyte binding IgM antibody and in vivo splicing of these regions to the IgM constant regions. In this embodiment the entire IgM molecule is cloned and expressed to make the RET Based Analyte Detector. In another preferred embodiment the heavy and light chain sequences of an analyte binding antibody may be cloned and fused to the IgM constant region sequences of the RET Based Analyte Detector by those knowledgeable in the art. Sequences that encode heavy and light chains of an antibody may be rationally designed to recognize an analyte. In other embodiments non-antibody analyte binding sequences may be fused to the IgM constant regions to create a RET Based Analyte Detector as long as they induce a conformational change in the IgM.

“Energy Donating Reagent” is contacted to the Physically Alterable Support. It donates the energy found in resonance energy transfer. The reagent is any molecule that emits the energy in the form of light. It may be a chemiluminescent molecule or a fluorescent molecule. The chemiluminescent molecule emits light intrinsically through a chemiluminescent reaction. The fluorescent molecule absorbs light from an extrinsic source and then reemits it. A preferred Energy Donating Reagent (EDR) would be a luciferase that in the presence of a Chemiluminescent Substrate produces light energy. The EDR may be a resonance energy donor when its emission spectrum overlaps with the absorption spectrum of an Energy Accepting Fluorescent Reagent (EAFR)

“Energy Accepting Fluorescent Reagent” is contacted to the Physically Alterable Support. It accepts the energy found in resonance energy transfer. It can include a protein or a fluorescent dye molecule. Proteins that accept energy from a light emitting molecule through resonance energy transfer and reemit it by fluorescence are fluorescent proteins. Types of fluorescent proteins include but are not limited to green fluorescent protein (GFP), red fluorescent protein (RFP), blue fluorescent protein (BFP), cyan fluorescent protein (CFP), and yellow fluorescent protein (YFP). The fluorescent proteins also include mutated versions of the wild type fluorescent proteins like Enhanced GFP (EGFP). The light that is emitted by the Energy Accepting Fluorescent Reagent has a longer wavelength than that generated by the Energy Donating Reagent. Fluorescent dyes may also accept energy from a light emitting molecule through resonance energy transfer and reemit it by fluorescence. The EAFR may be an resonance energy Energy Accepting Fluorescent Reagent when its absorption spectrum overlaps with the emission of an EDR.

“Bioluminescent Molecule” means any biological molecule involved in a chemiluminescent reaction. The reaction may be either catalytic or stoichiometric.

“Chemiluminescent Emission Spectrum” means the range of photon wavelengths emitted by the Chemiluminescent Molecule. The spectrum is frequently defined by the wavelength of highest intensity from a chemiluminescent reaction.

“Chemiluminescent Substrate” means a reactant required for a Chemiluminescent Molecule to produce a photon/light.

“Chemiluminescent Molecule” means any molecule that takes part in any chemiluminescent reaction; this includes but is not limited to a bioluminescent molecule.

“Chemiluminescent Reaction” means any chemical reaction that produces a photon without an input photon. The reactants may act either catalytically or stoichiometrically. In the case of a catalytic reaction, the catalyst converts a substrate(s) into a product(s) with the concomitant release of a photon. In the case of a stoichiometric reaction, two or more reactants are converted to product(s) and a photon.

“Antibody” or “Ab” or “Immunoglobulin” is a protein that binds specifically to a particular analyte and is capable of selectively binding to at least one of the epitopes of the protein or other analyteic substance used to obtain the antibodies and is derived wholly or in part from the immunoglobulin coding regions of the respective animal. Antibody molecules differ in their specificity by virtue of variability in the amino acid sequence of their “variable region domains”. Antibodies useful in a preferred embodiment can be either polyclonal or monoclonal antibodies. Antibodies of a preferred embodiment include functional equivalents such as antibody fragments and genetically-engineered antibodies, including single chain antibodies. Antibodies of a preferred embodiment also include chimeric antibodies that can bind to more than one epitope.

“Tag” means any domain on one molecule that facilitates its association with another molecule. In one embodiment, the tag is a peptide sequence that can be expressed as part of a Physically Alterable Support that can serve to immobilize the Physically Alterable Support to a solid support. A tag may also be used attach the Physically Alterable Support to the Energy Donating Reagent or the Energy Accepting Fluorescent Reagent. Additionally, the tag can be a chemical group that can be used for chemical immobilization. In one embodiment, the peptide sequence can encode a peptide tag for the recognition sequence for enzymes for associating non-proteinaceous molecules such as biotin or carbohydrates or any other post-translational modification of the protein. The association can be either covalent or non-covalent. The tag can be any peptide sequence that has these properties. A number of peptide sequences that have the properties of a Tag are known and can be used in a preferred embodiment. As a non-limiting example, the following peptide sequences can be tags of a preferred embodiment: a biotin accepting peptide sequence, hexa-His peptide, Strep-Tag, Strep-TagII, FLAG, c-myc, maltose binding protein (MBP), glutathione-S-transferase (GST), green fluorescent protein (GFP), red fluorescent protein (RFP), blue fluorescent protein (BFP), chitin binding protein, calmodulin binding protein (CBP), cellulose binding domain, S-tag, FIAsH, RsaA, and other similar types of peptide sequences having the ability to facilitate association with another molecule. In one embodiment, antibodies include as part of their coding sequences a biotin accepting peptide sequence that is a short amino acid sequence discovered by Schatz that allows the enzymatic attachment of biotin to a lysine residue within the tag. The Schatz biotin accepting peptide sequences are described in U.S. Pat. No. 5,723,584 issued on Mar. 3, 1998, U.S. Pat. No. 5,874,239 issued on Feb. 23, 1999, U.S. Pat. No. 5,932,433, issued on Aug. 3, 1999 and U.S. Pat. No. 6,265,552, issued July 2001. In general, biotin accepting peptide sequences have the following sequence: LeuXaa₁Xaa₂IleXaa₃Xaa₄Xaa₅Xaa₆LysXaa₇Xaa₈Xaa₉Xaa₁₀, (SEQ ID NO: 1) where Xaa₁ is any amino acid; Xaa₂ is any amino acid other than Leu, Val, Ile, Trp, Phe, or Tyr; Xaa₃ is Phe or Leu; Xaa₄ is Glu or Asp; Xaa₅ is Ala, Gly, Ser, or Thr; Xaa₆ is Gln or Met; Xaa₇ is Ile, Met, or Val; Xaa₈ is Glu, Leu, Val, Tyr, or Ile; Xaa₉ is Trp, Tyr, Val, Phe, Leu, or Ile; and Xaa₁₀ is any amino add other than Asp or Glu, wherein said biotinylation-peptide is capable of being biotinylated by a biotin ligase at the lysine residue adjacent to Xaa₆.

One embodiment of a biotin accepting peptide sequence is Gly Leu Asn Asp Ile Phe Glu Ala Gln Lys Ile Glu Trp His Glu (SEQ ID NO:2), and this sequence is referred to as the Avitag™. The peptide tag may, optionally, be appended to any part of the Physically Alterable Support. In one embodiment, the peptide tag is incorporated into a constant region of an immunoglobulin locus that is part of the Physically Alterable Support. Alternatively, the Physically Alterable Support can be covalently attached to any solid support without a Tag.

“Solid support” includes any suitable support for a binding reaction and/or any surface to which molecules may be attached through either covalent or non-covalent bonds. This includes, but is not limited to, membranes, plastics, paramagnetic beads, charged paper, nylon, Langmuir-Blodgett films, functionalized glass, germanium, silicon, silicon nitride, PTFE, polystyrene, gallium arsenide, gold and silver. Any other material known in the art that is capable of having functional groups such as amino, carboxyl, thiol or hydroxyl incorporated on its surface, is also contemplated. This includes surfaces with any topology, including, but not limited to, flat surfaces, spherical surfaces, grooved surfaces, and cylindrical surfaces e.g., columns. Multiple Physically Alterable Supports, each specific for a different analyte, may be attached to specific locations on the surface of a solid support in an addressable format to form an array, also referred to as a “microarray” or as a “biochip.”

### The General Method

One embodiment comprises: a Physically Alterable Support, an Energy Donating Reagent such as a luciferase and an Energy Accepting Fluorescent Reagent such as a green fluorescent protein. These reagents are combined through covalent or non-covalent interactions to form a single molecule. The Physically Alterable Support can be an antibody such IgM, antibody fragment, or antibody-like molecule prepared by any method known in the art, such as the preparation of monoclonal antibodies via the hybridoma method, the use of antibody phage display libraries, and so on. Conformational changes in a protein can alter both the distance between and orientation of associated Energy Donating Reagent and Energy Accepting Fluorescent Reagents. In the case where the Energy Donating Reagent is a bioluminescent protein and the Energy Accepting Fluorescent Reagent is a protein and resonance energy is transferred from the former to the later then the type of RET occurring is Bioluminescent Resonance Energy Transfer. BRET can detect changes of as little as about 1-2 Å when these two components are within about 100 Å of one another.

There are various ways in which the associated Energy Donating Reagent and Energy Accepting Fluorescent Reagent may be configured on the monomeric, pentameric or hexameric forms of the IgM. They may be attached covalently or non-covalently to the IgM. They may be expressed as fusions to the amino or carboxyl termini of the heavy or light chains IgM. One or more Energy Donating Reagents may be positioned at the center of the pentamer or hexamer while one or more Energy Accepting Fluorescent Reagents may be positioned on the light chains. One or more Energy Accepting Fluorescent Reagents may be positioned at the center of the pentamer or hexamer while one or more Energy Donating Reagents may be positioned on the light chains. Both the Energy Donating Reagent and the Energy Accepting Fluorescent Reagent may be positioned on the light chains. It is envisioned that any of these arrangements will generate a complex that will display a change in wavelength emission from the RET Based Analyte Detector upon conformational change. The amount of the reagents attached to arms may be modulated by co expressing in a cell line a vector that has unmodified light chain with respect to the Reagent. This will allow for the production of pentameric or hexameric IgMs on which only a fraction of the light chains possess a Reagent.

In this embodiment, analyte binding is detected indirectly by a physical alteration in the structure of the Physically Alterable Antibody, particularly analyte binding is detected indirectly by a conformational change in the structure of the IgM molecule. As described above, conformational changes may be detected by changes in the resonance energy state between an Energy Donating Reagent and an Energy Accepting Fluorescent molecule. The Energy Donating Reagent may be a chemiluminescent molecule such as a luciferase as is the case with BRET or it may be a dye that absorbs light form an extrinsic source such as a laser and then remits energy at a longer wavelength as is the case with FRET.

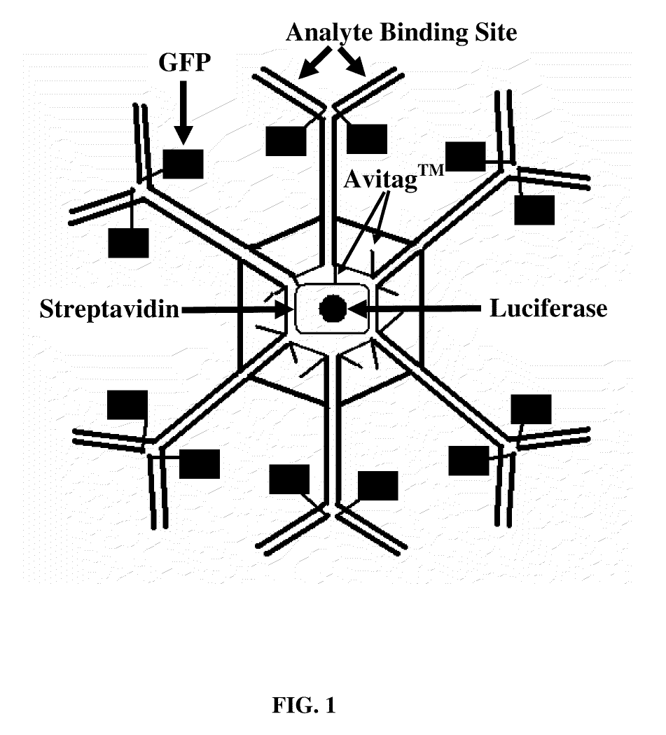

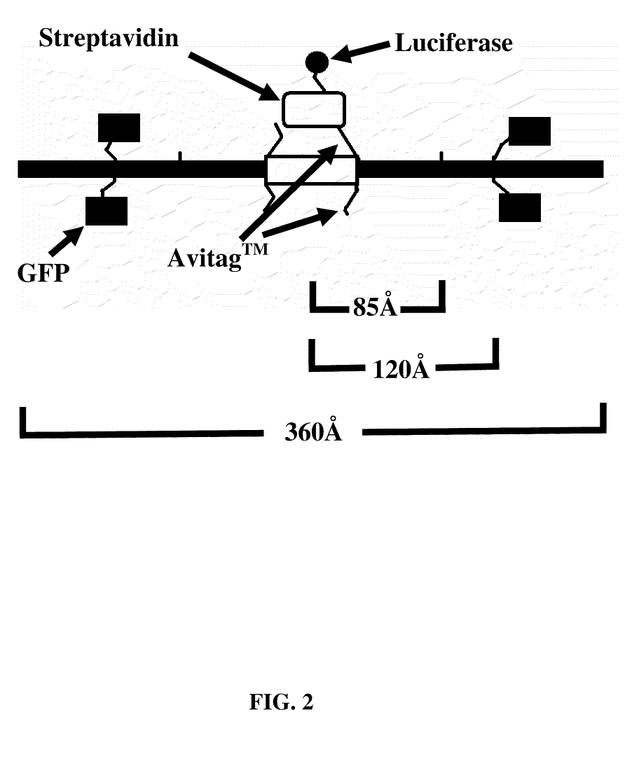

When the Physically Alterable Support is an IgM hexamer two vectors are made for its expression: 1) A vector is made for the expression of IgM antibody heavy chain that has the sequence coding for an Avitag™ operably linked to the coding sequence for the portion of IgM that can undergo physical alteration, such as the constant regions of the antibody's heavy chain. 2) A vector is made for the expression of the IgM antibody light chain that has the sequence coding for a green fluorescent protein operably linked to the coding sequence of the antibody's light chain variable region. In this embodiment, one or more luciferase proteins are attached to center of the IgM hexamer and green fluorescent proteins are attached to the IgM's variable regions. In the case of the hexameric form of the IgM there are twelve Energy Accepting Fluorescent Reagents or green fluorescent proteins per one hexameric molecule. (FIG. 1)

These two vectors are co-expressed in a single cell line to produce a hexameric form of the IgM with modified heavy and light chains. When the antibody is an IgM expressed by a hybridoma or B-cell, the antibody can be purified from this source directly. When the antibody is a non-IgM expressed by a hybridoma or B-cell, then the segment of the cDNA encoding the antibody hypervariable variable regions are cloned into the IgM expression vectors described above such that the variable regions are operably linked to the coding sequence for the portion of the Physically Alterable Support that can undergo conformational alteration, such as the IgM constant region. This constitutes the Modular Analyte Binding Region. The purified chimeric IgM possessing both the Avitag sequence and a fused green fluorescent protein is tested to confirm that that the recombinant IgM retains the expected analyte specificity and affinity. By “specificity,” it is meant that the IgM selectively binds the specified antigen or other analyte. Binding can be measured using a variety of methods known to those skilled in the art including immunoblot assays, immunoprecipitation assays, radioimmunoassays, enzyme immunoassays (e.g., ELISA), immunofluorescent antibody assays and immunoelectron microscopy. Antibodies that exhibit a specific binding at a level suitable for detection can be used as the Physically Alterable Antibody. The purified IgM and biotin are reacted with the biotin ligase enzyme to produce IgM molecules with one biotinylated Avitag™ per IgM constant region. In the case of the hexameric form of the IgM there are twelve biotinylated Avitag™s per one hexameric molecule. The IgM is immobilized on a streptavidin coated solid support via the biotinylated Avitag™s. The average distance between biotin binding sites on a streptavidin coated surface is about 20 Å. Avitag™ linkers that are about 50 Å in length should be sufficient for several of the Avitag™ to attach the IgM molecule to the surface but not long enough for all of the Avitag™s to bind to the streptavidin coated surface due to distance constraints. The remaining un-attached Avitag™s will be able to migrate through the central hole of the planar molecule.

In some embodiments, the Energy Donating Reagent is made as follows. The ability of tetrameric streptavidin to bind four molecules of biotin is exploited to produce a complex in which streptavidin acts as an intermediary to bind the luciferase and the IgM into a single complex. The coding sequence of a luciferase is operably linked to the coding sequence of the Avitag™. The chimeric luciferase is expressed in a suitable (bacterial or eukaryotic) system, purified and tested to confirm that it will function in a chemiluminescent reaction. The purified luciferase and biotin are reacted with the biotin ligase enzyme such that the Avitag™ part of the chimeric luciferase is biotinylated. The biotinylated luciferase is purified. The biotinylated luciferase is reacted with streptavidin in amounts such that only a fraction of the total biotin binding sites of the streptavidin are occupied by the biotinylated chimeric luciferase. The luciferase conjugated to streptavidin is added to immobilized biotinylated IgM fused to green fluorescent protein. The luciferase streptavidin molecule attaches to the available biotin molecules on the IgM. In the hexameric form of the IgM there are twelve biotinylated Avitag™s available for attachment to both a streptavidin solid support and to the luciferase streptavidin chimera. Only a portion of the biotinylated Avitag™s are occupied with attaching the IgM to the streptavidin coated support. Only a single biotinylated Avitag™ is needed to bind the luciferase streptavidin conjugate to the IgM. (FIGS. 1 and 2)

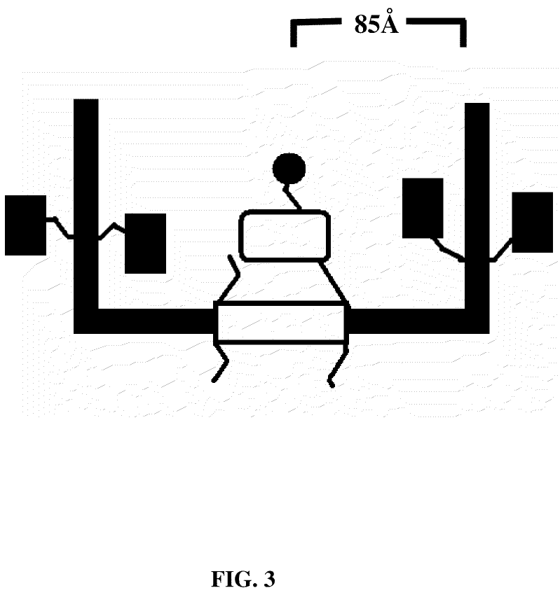

If the analyte of interest binds to the Physically Alterable Antibody, the molecule changes conformation in a fashion that is reflected as an increase in the resonance energy state that exists between the Energy Donating Reagent and the Energy Accepting Fluorescent Reagent. (FIG. 3) In this case, the Energy Donating Reagent is a luciferase and the Energy Accepting Fluorescent Reagent is a green fluorescent protein. The change in the resonance energy state is reflected in the amount of longer wavelength light emitted by the fluorescent protein relative to the amount of shorter wavelength light emitted by the luciferase when the luciferase performs a chemiluminescent reaction.

The method presented here includes the measurement of a physical change in an antibody molecule induced by the binding of analyte. Such a change would be independent of the nature of the analyte, and thus suited for simultaneous measurements of different analyte:antibody complexes. For example, when a number of different antibodies are immobilized on a slide and allowed to react with a sample, the analytes in this sample bind to their cognate receptors/antibodies immobilized on different regions of the slide. These antibodies with bound analyte can then be detected by change in the spectrum of light emitted from different regions of the slide when the bioluminescent reaction is initiated.

This embodiment provides a general method for detecting analyte-IgM antibody binding events. The method is independent of the specific analyte used, and relies instead on the general characteristic of IgM in which binding of specific analyte results in a dramatic conformational change in the IgM molecule, and that this change can be detected using changes in the resonance energy state that exists between a luciferase and fluorescent proteins that are arrayed on the IgM.

One embodiment provides detection of specific analyte binding to its cognate IgM antibody when the antibody is immobilized as discrete spots on a surface such as a streptavidin coated glass slide. The IgM as a component of the RET Based Analyte Detector is immobilized on the slide using existing or adapted technologies. The preferred method would use a genetically engineered attachment linker or tag that would allow the antibody to be tethered in a consistent orientation and allow sufficient flexibility for the antibody to undergo its conformational changes upon analyte binding. The same tag may serve to attach the Energy Donating Reagent to the IgM. For example, an IgM genetically engineered to contain an Avitag™ and extended flexible linker to the C-terminus of the Ig heavy chain will allow for robust, yet flexible tethering of the IgM to streptavidin coated surfaces. In the preferred embodiment the IgM will possess one Avitag™ per heavy chain. In a hexameric form of the IgM there will be twelve Avitag™s peptides available for biotinylation and attachment to either the streptavidin coated support or to the Energy Donating Reagent, in this case the luciferase streptavidin molecule. Linker length can be adjusted to ensure that only a fraction of these Avitag™ molecules will attach to a streptavidin-coated support due to constraints of distance. This will leave Avitag™s available to be bound to the Energy Donating Reagent. While one is sufficient, more than one Energy Donating Reagent may be attached.

The slide containing the immobilized RET Based Analyte Detector is then reacted with the analyte-containing test material, followed by appropriate washing to remove unbound material. Molecules of immobilized antibody that have bound specific analyte will have undergone the characteristic conformational change associated with analyte binding. The appropriate Chemiluminescent Substrate, such as coelenterazine, is then added. The luciferase catalyzes the production of light that has a spectrum of wavelengths that may be absorbed by the Energy Accepting Fluorescent Reagent attached to the variable region of the IgM. In a hexameric structure there are twelve fluorescent proteins attached.

In this method, the binding of analyte to the immobilized IgM is detected indirectly by virtue of the conformational change in the antibody structure. The decrease in distance between the luciferase and the fluorescent protein will be enough to significantly increase the transfer of energy from the luciferase to the fluorescent protein through resonance. The resulting increase in BRET is detectable as an increase in the amount of longer wavelength light that is emitted by the fluorescent proteins.

In this iteration of the method, the antibody conformation is detected by the ability of only the analyte-bound conformation of the IgM to produce longer wavelength light. Immobilized antibody that has bound its cognate analyte from the test material will move the fluorescent protein closer to the luciferase. Antibody spots that fail to bind analyte (heterologous antibody for which there is no cognate analyte in the test material, or if analyte is not present in the sample) are not conformationally altered, will remain in a planar configuration and have the fluorescent protein more than 85 Å from the light source and therefore not generate a detectable signal.

Any technology that can detect light and discriminate longer wavelength spectrums of light from shorter may be used to monitor signal.

### Making the RET Based Analyte Detector

This disclosure teaches a RET Based Detector for analyte binding, and methods of use thereof. The RET Based Analyte Detector is a single molecule consisting of an analyte specific Physically Alterable Antibody, an Energy Donating Reagent and an Energy Accepting Fluorescent Reagent. In this embodiment the RET Based Analyte Detector is made with four steps: 1) Construction of a modified IgM hexamer with Avitag™ linkers attached to the IgM heavy chain and Energy Accepting Fluorescent Reagent attached to the IgM light chain; 2) Introduction of analyte specificity to the IgM; 3) Production of the Physically Alterable Support and Energy Accepting Fluorescent Reagent; 4) Addition of the Energy Donating Reagent.

1) Construction of the Modified IgM Hexamer with Avitag™ Linkers Attached to the IgM Heavy Chain and Energy Accepting Fluorescent Reagent Attached to the IgM Light Chain.

Manipulation of DNA sequences and generation of viral vectors is performed using methods known to those persons in the art. The overall strategy for the expression of the modified IgM is to subclone a) the heavy chain sequence with added Avitag sequence and b) light chain sequence with added green fluorescent protein sequence into retroviral expression vectors. Sequences that confer recognition against a specific analyte are then introduced into these vectors. Two recombinant retroviral strains are produced; one that expresses the analyte specific IgM heavy chain with Avitag™ attached and another that expresses the analyte specific IgM light chain with the GFP attached. These two retroviral strains are used to co-infect a cell line suitable for expression. Cells that co express the two separate sequences will be screened.

Specifically, the retroviral vectors used contain the sequences needed for the expression of a transcript that can be packaged into retroviral virions. The vector is designed to express an inserted gene as well a fluorescent protein off of the 5′retroviral LTR promoter. The inserted gene is 5′ to the yellow fluorescent protein (YFP) and between the two is an internal ribosome entry site (IRES) element. The bicistronic RNA that is produced allows for the separate translation of the two proteins from the same transcript. The heavy chain sequence is inserted into the vector. A linker sequence followed by Avitag™ is added in frame to the 3′ end of the heavy chain sequence to generate a final added length of 75 nucleotides.

A separate vector designed to express the light chain of IgM is made. The sequence for the green fluorescent protein (GFP) is fused to the 3′ end of the light chain sequence with 10 amino acid linker between the two.

### 2) Introduction of Analyte Specificity to the IgM

The nucleotide sequence that provides the specificity to the IgM for recognition of an analyte can be obtained from any one of several sources. Animals can be immunized with a particular analyte. The predominant Ig types from hyperimmune serum or by monoclonal antibody-producing hybridomas are IgG. General methods for making suitable IgM Physically Alterable Support reagents from these sources are described herein. IgM producing cell lines can be produced de novo, e.g., from immunized animals by standard cell fusion techniques or by immortalization of IgM producing B-cells with Epstein-Barr virus, and the resulting cell lines screened for those producing IgM with the desired analyte specificity. In one embodiment, existing hybridomas producing Ig with an analyte specificity of interest, but of an isotype other than IgM, can be used to create cell lines producing chimeric IgM having the Ig heavy- and Ig light-chain variable regions (and therefore the analyte binding affinity and specificity) derived from the original Ig molecule and IgM-derived mu-heavy chain constant regions. For example, in one embodiment, mRNA from an IgG-producing hybridoma, is used in RT-PCR to amplify the heavy and light-chain variable regions of the expressed Ig light and heavy chains, and cloned into respective light chain and mu heavy chain expression vectors. The vectors contain an intact cDNA of the kappa (or lambda) light chain, or the mu heavy chain constant regions, such that the respective variable regions of each chain can be appended to other respective constant regions. The resulting constructs are co-expressed (with or without expression of the J-chain) in appropriate cell lines (e.g., non-Ig producing myeloma) for expression. Alternatively, the IgG (or other) producing hybridoma can be manipulated genetically to produce IgM with the same variable regions and having the same analyte specificity and affinity. This is accomplished by targeted gene replacement in which the mu heavy chain constant region is inserted in the chromosome juxtaposed to the respective rearranged and expressed heavy chain variable region gene. The resulting construct expresses a chimeric IgM having a heavy chain that is derived from (a) the original variable region and (b) the introduced mu constant regions. This method can also utilize the variable regions or analyte-binding domains derived from any class of antibody (e.g., IgY, IgG, IgM, IgA, IgE,), or immunoglobulin-like cell surface receptor molecules (e.g., T-cell receptors and other cell surface receptor molecules), or other analyte-binding proteins, from a wide array of mammalian species (including but not limited to goats, rabbits, mice, rats, horses, llamas), or from avian species such as chicken, or from fish species (including but not limited to sharks and zebrafish).

In one embodiment, antibody-like molecules, such as single-chain variable region fragment (scFv) antibodies from phage display, may also be “converted” to authentic IgM and have the analyte specificity of the original antibody-like molecule. For example, the respective variable regions of a single chain antibody gene of interest may be cloned and inserted into appropriate light chain and mu heavy chain expression vectors.

IgM may be expressed in a variety of configurations (e.g., various oligomeric forms, including monomers, pentamers, hexamers, and forms with or without the associated J-chain, and forms with or without an associated “secretion component” derived from the polymeric immunoglobulin receptor, and membrane-bound surface form of IgM, sIgM, or the soluble form of IgM). In certain applications, one or another of these forms may be most applicable. While in most cases, the constant regions of antibody molecules do not appear to undergo physical change (allosteric or conformational) upon analyte binding, IgM is an exception: IgM is a pentamer (or in some cases a tetramer or hexamer) of dimers each with an antibody combining site of one heavy and one light chain, all joined by a “J-chain”. Thus, these molecules are decavalent for analyte combining sites. The best evidence indicates that occupancy of two or more analyte binding sites on this molecule induces a change such that binding sites for complement factor C1 (or sub-factor C1q) are exposed. As the affinity of IgM for analyte is generally low, occupancy of two or more analyte binding sites normally occurs only when the analyte is multivalent. However, it is possible to select for IgM of high affinity or to introduce analyte combining sites (variable regions) of high affinity from other classes of immunoglobulins into IgM by means of recombinant DNA techniques. The cell line, including a mutagenized hybridoma cell line, engineered to produce the IgM Physically Alterable Support Reagent can be made with or without the J chain. The J chain could be produced by co-transfection of the J-chain producing vector with the IgM expression vector, could be co-expressed a different promoter on the expression vector that produces the IgM, could be produced by a combination of chromosomally integrated and cytoplasmic expression replicon, or by any method that is currently used to express multiple or multi-subunit proteins.

### 3) Production of the Physically Alterable Support and Energy Accepting Fluorescent Reagent

Two recombinant retroviral strains are produced. The vector in plasmid DNA form that contains the heavy chain sequence fused to the Avitag sequence and which possesses the variable region sequences that confer analyte specificity is transiently transfected into an amphotropic retroviral packaging cell line. Recombinant virions are harvested after 24 hr. The vector in plasmid DNA form that contains the light chain sequence fused to GFP sequence and which possesses the variable region sequences that confer analyte specificity is transiently transfected into an amphotropic viral packaging cell line. Recombinant virions are harvested after 24 hr. Both of these recombinant viral strains are used to co infect a mouse pituitary cell line. The population of infected cells is subjected to fluorescence activated cell sorting (FACS). The cells that produce both GFP and YFP are harvested. These cells are co-expressing the modified heavy and light chains of the IgM. In the absence of expression of the ancillary J chain protein the resulting molecule produced is the hexameric form. The IgM is secreted into the tissue culture media. The IgM is then purified and tested to confirm that it recognizes the analyte to be detected.

IgM multimers have been expressed in the various cell lines including glial cells, pituitary cells, and B cell lines. Depending on the cell lines used to express the IgM there may be some expression of a J chain minus pentamer so it may be necessary to use a gel filtration step to ensure a homogeneous preparation of hexamer. The hexamer produced this way will have twelve Avitag™ linkers hanging off of the carboxyl terminus of the twelve heavy chains. These linkers will be located in central hole of the planar hexamer. This hole is about 60 Å in diameter and should be of sufficient size for the linkers to migrate back and forth through. Because the planar form of the IgM multimer is symmetrical the linkers should not have a preference for either side of the plane. Purified IgM and biotin are reacted with biotin ligase to facilitate the covalent attachment of biotin to the Avitag™.

4) The Energy Donating Reagent is made as follows. The Avitag™sequence is fused to the 3′ end of the sequence of luciferase of the copepod, Gaussia princeps. The chimeric luciferase is expressed in a suitable (bacterial or eukaryotic) system, purified and tested to confirm that it will function in a chemiluminescent reaction. The purified luciferase and biotin are reacted with the biotin ligase enzyme such that the Avitag™ part of the chimeric luciferase is biotinylated. The biotinylated luciferase is purified. The biotinylated luciferase is reacted with streptavidin in amounts such that only a fraction of the total biotin binding sites of the streptavidin are occupied by the biotinylated chimeric luciferase.

### 5) Assembly of the RET Based Analyte Detector.

The luciferase conjugated to streptavidin is added to biotinylated IgM fused to green fluorescent protein. The luciferase streptavidin molecule attaches to the available biotin molecules on the IgM. In the hexameric form of the IgM there are twelve biotinylated Avitag™s available for attachment to both a streptavidin solid support and to the luciferase streptavidin chimera. Only a portion of the biotinylated Avitag™s are occupied with attaching the IgM to the streptavidin coated support. Only a single biotinylated Avitag™ is needed to bind the luciferase streptavidin conjugate to the IgM. Alternatively, the RET Based Analyte Detector may be assembled in solution with no attachment to a solid support.

### Detection

The specific analyte to be detected is introduced into the media containing the Detector. The variable region sequences of the Physically Alterable Support or IgM hexamer bind the epitopes of the analyte inducing a conformational change in the IgM molecule. In the condition where the IgM is bound to a solid support the Detector is washed to remove unbound analyte. The Chemiluminescent Substrate, coelenterazine is added to the Detector initiating an energy generating reaction with the luciferase. Alternatively, a wash step may be omitted and coelenterazine may be directly added to the analyte Detector mix. Alternatively, the Detector may be unattached to a solid support and exist in solution. It can react with analyte in solution with no wash step. Coelenterazine may be added to the analyte Detector mix. If the analyte of interest binds to the Physically Alterable Antibody, the molecule changes conformation that is reflected as a change in the resonance energy state that exists between the Energy Donating Reagent and the Energy Accepting Fluorescent Reagent. In this case, the Energy Donating Reagent is a luciferase and the Energy Accepting Fluorescent Reagent is a green fluorescent protein. The change in the resonance energy state is reflected in the amount of longer wavelength light emitted by the fluorescent protein relative to the amount of shorter wavelength light emitted by the luciferase when the luciferase performs a chemiluminescent reaction. This change in fluorescence will occur regardless of whether the RET Based Analyte Detector is attached to a solid support or is in solution phase.

Any suitable detector for the fluorescence change is envisioned, which will collect light and discriminate shorter wavelength light from longer wavelength light. These detectors include, but are not limited to: spectrofluorimeter, fluorimeter, and luminometer equipped with filters. In one embodiment, the method for the detection of RET using a luminometer comprises removing the shorter wavelength light produced by the luciferase by including a non-fluorescing quenching dye such as [4-((4-dimethylamino)phenyl)azo)benzoic acid] (DABCYL) in the light producing reaction solution. DABCYL absorbs light with a maximum at 480 nm and permits the green light of the GFP to pass to the luminometer detector.

### Uses

The disclosure teaches a general method for detecting binding. Since binding reactions, such as, antibody recognition of analytes, is widely used in the quantitation of proteins and other molecules, the detection of such complexes is important for many applications, including, but not limited to, proteomics and diagnostic measurements of proteins and other molecules in bodily tissues and fluids.

All patents and publications referred to herein are expressly incorporated by reference in their entirety. The following examples serve to illustrate certain preferred embodiments and aspects of the present invention and are not to be construed as limiting the scope thereof.

### EXAMPLES

### Example One

One preferred embodiment of the RET Based Analyte Detector is the IgM schematically depicted in FIGS. 1, 2 and 3. A general approach for making this molecule is described here. An IgM expression vector is engineered by those knowledgeable in the art to have the Avitag™ sequence at the end of a linker 30 amino acids in length fused to the carboxyl terminus of the constant region of the IgM heavy chain. The vector also has the sequence for the GFP fused to the carboxyl terminus of the IgM light chain variable region.

Hybridomas producing high affinity antibodies for a specific analyte are selected by standard techniques. The variable regions of the IgG heavy and light chains expressed by this hybridoma are cloned and introduced into the IgM expression vectors with the Avitag™ fused heavy chain and GFP fused light chain respectively. These DNA constructs are then introduced into a cell appropriate for their expression and secretion, such as a myeloma cell line. The IgM is expressed in the absence of the J chain such that hexameric IgM is assembled in the cells. The fusion IgM molecules produced by these cells have twelve Avitag™s and twelve GFPs per hexameric molecule. The modified IgM reagent is purified by standard means, biotinylated and immobilized on a streptavidin coated well of a 96 well microtiter plate. The chimeric molecule consisting of the biotinylated luciferase from Gaussia princeps and streptavidin are added to the IgM molecule to assemble the RET Based Analyte Detector. The well is washed with a suitable buffer solution to remove unbound Reagent.

A sample (tissues or body fluids) is then allowed to react in the well and analyte in the sample binds to the antibody. The 96 well microtiter plate is placed on a light detector such as a luminometer with a filter that removes light lower than 500 nm. Coelenterazine is then added to the slide and light is measured. The antibody:analyte complexes are then detected as an increase in the green light detected over background.

A version of the Detector molecule described above was partially constructed and examined as follows.

Cloning of Anti-TNP IgM Heavy and Light Chain with AviTag and EGFP

Messenger RNA was isolated from a lymphocyte-derived anti-Trinitrophenol (TNP) IgM expressing cell line, SP6/HL (Schulman). An RT-PCR reaction was used to amplify and generate a cDNA of the heavy chain (H chain) with a Gly₄Ser₄ linker motif encoding sequence in frame at the 3′end of the amplified product. A second round of PCR amplification, was used to fuse the Avitag™sequence in frame to the 3′ end of the Gly₄Ser₄ linker.

The light chain of anti-TNP IgM was fused to an Enhanced Green Fluorescent Protein (EGFP) at the carboxy terminus. The mRNA isolated above was RT-PCR amplified with a sense and an antisense primer containing (Gly₄Ser)₃ coding sequence at the 3′terminus. Another PCR product for EGFP coding sequence was generated from a DNA plasmid. Both PCR products were ligated such that EGFP is fused in-frame to the linker of light chain. This strategy resulted in a DNA ligated product which encoded light chain with a linker followed by EGFP coding sequence.

The DNA fragments for the modified Heavy and Light Chain constructs were then cloned into retroviral vectors for expression. Plasmid DNA for Light Chain expression was transfected into Phoenix eco cells (ATCC). Forty-eight hours later, virus was harvested form the supernatants and J558L myeloma cells were transduced by spinfection. Forty-eight hours post transduction, fluorescence activated flow cytometry was used to check for expression of the EGFP fusion in the J558L cells. The J558L cell line expresses the J chain and is known to produce a 1:1 mix of pentamers and hexamers when stably expressing the IgM H and L chains. One of the positive cell lines was sorted for high EGFP expression and the sorted cells were grown in culture and western blots were performed to check for expression of the light chain EGFP fusion.

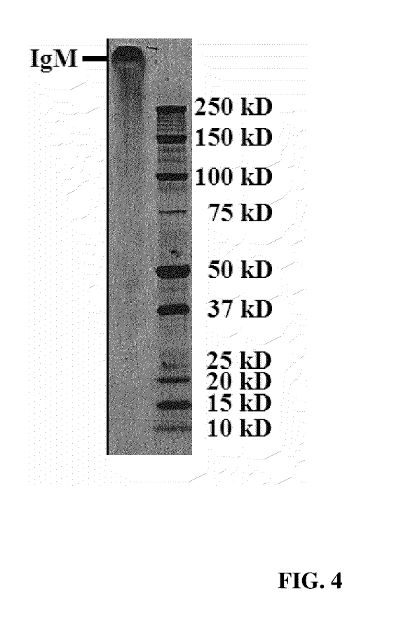

A retroviral vector for the expression of the modified heavy chain was also made in the same fashion as for the light chain. The Thy1.1 cell surface antigen was engineered to be coexpressed by the vector. The retrovirus was used to infect the modified Light chain expressing cells. Forty-eight hours later fluorescently labeled antibodies against the Thy1.1 were used in conjunction with fluorescence activated flow cytometry to screen for heavy chain transductants. Post transduction, the cells were sorted for high EGFP and Thy1.1 expression. The supernatants of cells expressing the EGFP and Thy1.1 were analyzed by western blots with goat anti-mouse IgM (μ chain) to determine if IgM is produced and assembled into a high molecular weight pentamer/hexamer. The expected molecular weight for the hexamer is approximately 1000 kDa. Separation on a non-reducing gel and western blot detection indicate a high molecular weight IgM is being made and secreted. (FIG. 4)

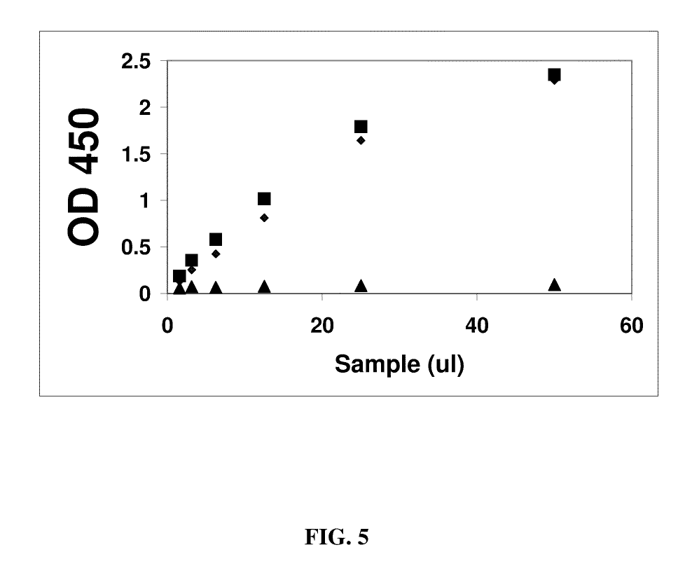

To determine if the IgM found in the supernatants recognizes antigen, an antigen capture ELISA assay was performed. (FIG. 5) Wells of a 96 High binder ELISA plate (Costar) were coated with 2 ug of TNP conjugated to ovalbumin. The wells were blocked with BSA (0.3% in PBS) and cell supernatants reacted to the TNP-ovalbumin (antigen). Following washing with PBS, bound IgM was detected with anti-mouse IgMμ-Horse Radish peroxidase (HRP) conjugate using an HRP enzymatic/colorimetric assay. The data in FIG. 5 indicate that the modified IgM binds TNP-ovalbumin in the same fashion as unmodified wild type IgM and consistent with the notion that it is functional.

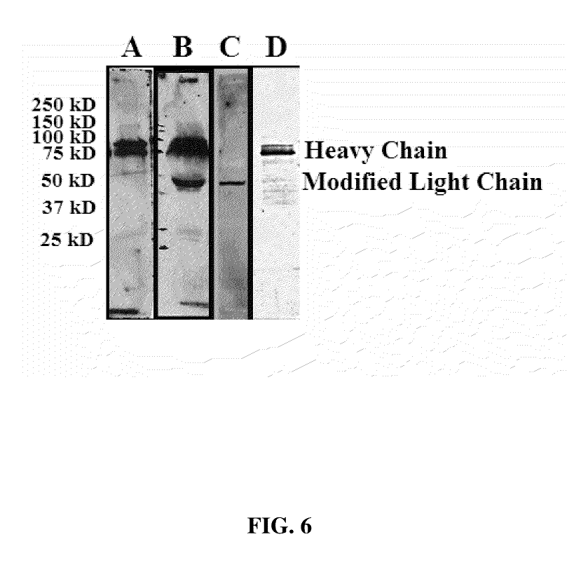

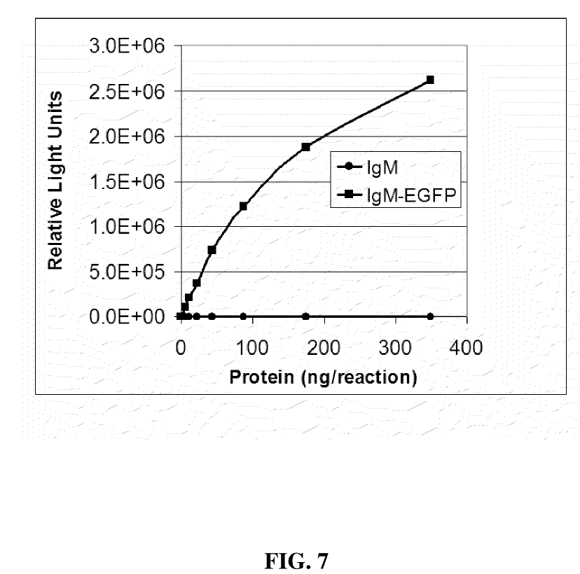

IgM from pooled tissue culture supernatants was purified using HiTrap™ IgM Purification HP Column (GE Healthcare). Biotin ligase, biotin, ATP and MgCl₂ were reacted with purified IgM to biotinylate the AVITAG peptide fused to the heavy chain. The purified material was resolved by SDS PAGE, transferred to nitrocellulose then examined with a variety of probes to determine if it 1) possesses the L Chain fused to EGFP and 2) is biotinylated as would be expected if the Avitag is fused to the H Chain. (FIG. 6)

Modified IgM (Lane C) and unmodified IgM (Lane A) were first probed with rabbit anti-EGFP primary antibody, which was then probed with goat anti-rabbit conjugated to HRP secondary antibody. These blots were visualized with HRP chemiluminescent substrate. A band appears with the modified IgM (Lane B) of a molecular weight consistent with that of a fusion of L Chain with EGFP (49 kD) recognized by the anti-EGFP antibody.

These blots were then probed with anti-IgM (μ chain) conjugated to HRP. The blots were visualized with HRP chemiluminescent substrate. Normal H chain (recognized by the anti-μ chain antibody) is present in both lanes as expected. No band appears in Lane A at the molecular weight for the L Chain plus EGFP.

A blot of modified IgM (Lane D) was probed with streptavidin conjugated to alkaline phosphatase. After was washing the alkaline phosphatase was visualized by reacting with enzymatic stain. A band the size of the H chain in this lane is stained indicating that the strepatavidin probe bound to biotinylated IgM. This indicates that the Avitag™ is present an biotinylated as expected if fused to the H Chain.

The chemiluminescent reaction of the Gaussia princeps luciferase is significantly quenched when performed in the presence of DABCYL dye as detected by a Luminometer which has a broad spectral detection range. This is because the emission spectrum of the luciferase is about the same as the absorption spectrum of DABCYL the peaks of both being about 480 nm. It is expected that when the same reaction is performed in the presence of increasing amounts of EGFP there will be an increase in the amount of light detected by the Luminometer. More energy is transferred from the luciferase to EGFP which will in turn emit longer wavelength (>500 nm) light that will pass through the DABCYL dye. This will allow for the sensitive detection of an RET using a Luminometer. To test this, the following experiment was performed. Purified modified IgM was added in increasing amounts to fixed amounts of luciferase and DABCYL dye. FIG. 7 shows that in the presence of DABCYL dye at sufficiently concentrations the light produced by luciferase is quenched by 99.99%. However, as IgM conjugated to EGFP is added to the reaction as much of 45% of the light is recovered. This is consistent with the notion that IgM plus EGFP will significantly shift the light produced in the reaction to a longer wavelength.

### Example Two

An IgM expression vector is engineered to have the Avitag sequence at the end of a linker 30 amino acids in length fused to the carboxyl terminus of the constant region of the IgM heavy chain. The vector also has the sequence for the GFP fused to the carboxyl terminus of the IgM light chain variable region.

Hybridomas producing high affinity antibodies for a specific analyte are selected by standard techniques. The variable regions of the IgG heavy and light chains expressed by this hybridoma are cloned and introduced into the IgM expression vectors with the Avitag™ fused heavy chain and GFP fused light chain respectively. These DNA constructs are then introduced into a cell appropriate for their expression and secretion, such as a myeloma cell line. The IgM is expressed in the absence of the J chain such that hexameric IgM is assembled in the cells. The fusion IgM molecules produced by these cells have twelve Avitag™s and twelve GFPs per hexameric molecule. The modified IgM reagent is purified by standard means, biotinylated and added to a tube. A chimeric molecule consisting of the biotinylated luciferase from Gaussia princeps and streptavidin are added to the buffer where a portion is conjugated to the IgM molecule to assemble the RET Based Analyte Detector. No wash step is included.

A sample (tissues or body fluids) is then added directly to tube where the analyte in the sample binds to the Detector. The tube is placed in a detector equipped with a filter that removes light lower than 500 nm. Coelenterazine is then added to the tube and light is measured. The antibody:analyte complexes are then detected as an increase in the green light detected over background. Forster's law of RET states that the degree of resonance energy transfer between the energy donor and energy Energy Accepting Fluorescent Reagent is inversely proportional to the distance between the two molecules to the sixth power. Because of the highly stringent requirement for close proximity between the energy donor and energy Energy Accepting Fluorescent Reagent in BRET, only luciferase that is in contact with the IgM will enter into a resonance energy state with the GFPs bound to the IgM. Luciferase that is not contacted to the IgM plus GFP will only generate blue light. This light will be filtered out. Thus a wash step is not required in order to detect analyte.

### Example Three

This example uses the same procedure as described in Example One, however, the locations of the luciferase and the GFP have been switched. An IgM expression vector is engineered to have the Avitag™sequence at the end of a linker 30 amino acids in length fused to the constant region of the IgM heavy chain. The vector also has the sequence for the Gaussia princeps luciferase protein fused to the light chain variable region.

Hybridomas producing high affinity antibodies for a specific analyte are selected by standard techniques. The variable regions of the IgG heavy and light chains expressed by this hybridoma are cloned and introduced into the IgM expression vectors with the Avitag™ fused heavy chain an luciferase fused light chain respectively. These DNA constructs are then introduced into a cell appropriate for their expression and secretion, such as a myeloma cell line. The IgM is expressed in the absence of the J chain such that hexameric IgM is assembled in the cells. The fusion IgM molecules produced by these cells have twelve Avitag™s and twelve luciferases per hexameric molecule. The modified IgM reagent is purified by standard means, biotinylated and immobilized on a streptavidin coated well of a 96 well microtiter plate. A chimeric molecule consisting of the GFP and streptavidin are conjugated to the IgM molecule to assemble the RET Based Analyte Detector. The well is washed with a suitable buffer solution to remove unbound Reagent.

A sample (tissues or body fluids) is then allowed to react on the surface of the slide and analyte in the sample binds to the antibody. The 96 well microtiter plate is placed on a light detector such as a luminometer with a filter that removes light lower than 500 nm. Coelenterazine is then added to the slide and light is measured. The antibody:analyte complexes are then detected as an increase in the green light detected over background.

It is to be understood that the foregoing general description and the detailed description are exemplary and explanatory only and are not restrictive of certain embodiments, as claimed.

What is claimed is:

1. A method for detection of binding, comprising:
a) providing a Physically Alterable Support,
b) providing a analyte, wherein the Physically Alterable Support specifically binds to the analyte;
c) contacting the Physically Alterable Support with an Energy Donating Reagent and an Energy Accepting Fluorescent Reagent;
d) detecting a change in the resonance energy state between the Energy Donating Reagent and an Energy Accepting Fluorescent Reagent, whereby binding of the Physically Alterable Support to the analyte is detected.

2. The method of claim 1, wherein the detection is quantitative.

3. The method of claim 1, wherein the Physically Alterable Support comprises
a) an analyte binding site;
b) a domain that becomes physically altered upon binding of analyte to the analyte binding site: and,
c) optionally, a site for coupling the binding reagent to a solid support.

4. The method of claim 1, wherein the Physically Alterable Support comprises an antibody selected from the group consisting of monomeric IgM, oligomeric IgM, an Fab fragment, an F(ab)₂ fragment, a genetically engineered antibody and a chimeric antibody.

5. The method of claim 1, wherein the Physically Alterable Support comprises a tag for coupling the Physically Alterable Support to a solid support.

6. The method of claim 5, wherein the tag is selected from the group consisting of biotin accepting peptide sequence, hexa-His peptide, Strep-Tag, Strep-TagII, FLAG, epitope tag, maltose binding protein (MBP), glutathione-S-transferase (GST), green fluorescent protein (GFP), red fluorescent protein (RFP), blue fluorescent protein (BFP), chitin binding protein, calmodulin binding protein (CBP), cellulose binding domain, S-tag, FIAsH, RsaA, and sortase recognition sequence.

7. The method of claim 6, wherein the biotin accepting peptide sequence is LeuXaa₁Xaa₂IleXaa₃Xaa₄Xaa₅Xaa₆LysXaa₇Xaa₈Xaa₉Xaa₁₀, where Xaa₁ is any amino acid; Xaa₂ is any amino acid other than Leu, Val, Ile, Trp, Phe, or Tyr; Xaa₃ is Phe or Leu; Xaa₄ is Glu or Asp; Xaa₅ is Ala, Gly, Ser, or Thr; Xaa₆ is Gln or Met; Xaa₇ is Ile, Met, or Val; Xaa₈ is Glu, Leu, Val, Tyr, or Ile; Xaa₉ is Trp, Tyr, Val, Phe, Leu, or Ile; and Xaa₁₀ is any amino add other than Asp or Glu, wherein said biotinylation-peptide is capable of being biotinylated by a biotin ligase at the lysine residue adjacent to Xaa₆.

8. The method of claim 7, wherein the biotin accepting peptide sequence is SEQ ID NO:2.

9. The method of claim 7, wherein said biotinylation sequence has been biotinylated by a biotin ligase.

10. The method of claim 1, wherein detecting a conformational change in the Physically Alterable Support to the analyte is selected from the group consisting of fluorescence emission, Raman shift spectroscopy, Fluorescence Resonance Energy Transfer (FRET), Bioluminescent Resonance Energy Transfer (BRET), Surface Plasmon Resonance, and Atomic Force Microscopy.

11. The method of claim 1, wherein the Energy Donating Reagent is a bioluminescent reagent.

12. The method of claim 11, wherein the bioluminescent reagent is luciferase.

13. The method of claim 12, wherein the luciferase performs a chemiluminescent reaction.

14. The method of claim 13, wherein coelenterazine is a substrate for the luciferase.

15. The method of claim 1, wherein the Energy Accepting Fluorescent Reagent is green fluorescent protein.

16. A composition comprising a Physically Alterable Support, an Energy Donating Reagent, and an Energy Accepting Fluorescent Reagent.

17. A microarray comprising a plurality of compositions according to claim 16 at specific locations on the surface of a solid support in an addressable format.

18. A method for detecting binding, comprising:
a) preparing a microarray according to claim 17;
b) providing a sample suspected of analyte containing a analyte, wherein the Physically Alterable Support specifically binds to the analyte;
c) contacting the Physically Alterable Support with an Energy Donating Reagent and an Energy Accepting Fluorescent Reagent;
d) detecting a change in the resonance energy state between the Energy Donating Reagent and an Energy Accepting Fluorescent Reagent, whereby binding of the Physically Alterable Support to the analyte is detected.

19. A method for detecting an analyte in a sample, comprising:
a) providing a detector comprising:
i) a Physically Alterable Support comprising a Modular Analyte Binding Region;
ii) an Energy Donating Reagent; and
iv) Energy Accepting Fluorescent Reagent;
b) contacting the sample with the analyte sensor, wherein a RET interaction between the Energy Donating Reagent and the Energy Accepting Fluorescent Reagent occurs when the analyte binding moiety binds the analyte in the sample, thereby producing an optical signal in response to binding the analyte; and
(c) detecting the optical signal, thereby determining the presence of the analyte in the sample.

20. The method of claim 19, wherein the detection is quantitative.

21. The method of claim 19, wherein the Physically Alterable Support further comprises a domain that becomes physically altered upon binding of analyte to the analyte binding site; and, optionally, a site for coupling the binding reagent to a solid support.

22. The method of claim 19, wherein the Physically Alterable Support comprises an antibody selected from the group consisting of monomeric IgM, oligomeric IgM, an Fab fragment, an F(ab)₂ fragment, a genetically engineered antibody and a chimeric antibody.

23. The method of claim 19, wherein the Physically Alterable Support comprises a tag for coupling the Physically Alterable Support to a solid support.

24. The method of claim 23, wherein the tag is selected from the group consisting of biotin accepting peptide sequence, hexa-His peptide, Strep-Tag, Strep-TagII, FLAG, epitope tag, maltose binding protein (MBP), glutathione-S-transferase (GST), green fluorescent protein (GFP), red fluorescent protein (RFP), blue fluorescent protein (BFP), chitin binding protein, calmodulin binding protein (CBP), cellulose binding domain, S-tag, FIAsH, RsaA, and sortase recognition sequence.

25. The method of claim 24, wherein the biotin accepting peptide sequence is LeuXaa₁Xaa₂IleXaa₃Xaa₄Xaa₅Xaa₆LysXaa₇Xaa₈Xaa₉Xaa₁₀, where Xaa₁ is any amino acid; Xaa₂ is any amino acid other than Leu, Val, Ile, Trp, Phe, or Tyr; Xaa₃ is Phe or Leu; Xaa₄ is Glu or Asp; Xaa₅ is Ala, Gly, Ser, or Thr; Xaa₆ is Gln or Met; Xaa₇ is Ile, Met, or Val; Xaa₈ is Glu, Leu, Val, Tyr, or Ile; Xaa₉ is Trp, Tyr, Val, Phe, Leu, or Ile; and Xaa₁₀ is any amino add other than Asp or Glu, wherein said biotinylation-peptide is capable of being biotinylated by a biotin ligase at the lysine residue adjacent to Xaa₆.
